(19) United States
(12) Patent Application Publication
(10) Pub. No.: US 2015/0150483 A1
(43) Pub. Date: Jun. 4, 2015

(54) DEVICE, METHOD AND SOFTWARE FOR MEASURING EXHALATION CAPACITY

(71) Applicant: Dan-Luis Mangell, Stockholm (SE)

(72) Inventor: Dan-Luis Mangell, Stockholm (SE)

(73) Assignee: POND HEALTH CARE INNOVATION AB, Stockholm (SE)

(21) Appl. No.: 14/405,857

(22) PCT Filed: Jun. 7, 2013
(86) PCT No.: PCT/SE2013/050650

§ 371 Date: Dec. 5, 2014

(30) Foreign Application Priority Data
Jun. 8, 2012 (SE) 1250597-0

(51) Int. Cl. A61B 5/085; A61B 5/097; A61B 5/00; A61B 5/09; A61B 5/087

(52) U.S. Cl. CPC A61B 5/085; A61B 5/09; A61B 5/0873; A61B 5/0004; A61B 5/097; A61B 5/6898; A61B 5/742; A61B 5/0026; A61B 5/7278

(57) ABSTRACT

Measurement device (1) for measuring the exhale capacity of a patient, includes a main body (2) with a through hole (3) with a blowing device (17) which has a rotation device (20) which is put in rotary motion through blowing, with a rotary speed depending on the intensity of the blowing, and the rotation of which is sensed by an optical sensor (6). The measuring device includes a cable (11) which is connected to the main body of the measurement device, and with a free end (11b) which is arranged to be connected to an audio input of a handheld general purpose computer unit (14), and in that the measurement device is arranged to, to the handheld computer unit, continuously deliver an analogue electric signal which carries information about rotary speed or air flow. A piece of software and a method are also described.

The present invention relates to a device, a method and a software product for measuring the exhale capacity of a patient.

Several patient categories have a need for occasionally or regularly measuring their exhale capacity. This goes for instance for COPC patients (Chronic Obstructive Pulmonary Disease) and asthma patients. Conventionally, such measurements are performed using a so called spirometer. Hereby, the patient blows, with maximum force, through a blowing tube, in the end of which there is a small turbine or propeller which is set in motion by the air stream through the tube. The tube itself, which is usually disposable, is inserted into a hole in the spirometer instrument, which optically reads the rotary speed of the turbine. This way, the instrument indirectly measures how forcefully the patient can blow, and as a result the exhale capacity of the patient.

Such tubes and spirometer instruments are marketed, for instance, by the company MIR (Medical International Research) in the USA.

One problem is that such instruments are expensive. It would be desirable for a larger group of patients to obtain simple and cheap access to measurement instruments for exhale capacity, even for private use outside the hospital, such as at home. By continuously and frequently supervise the development of the exhale capacity over time for broader groups of patients, serious states of illness can be discovered and treated earlier.

The present invention solves the above described problems. Hence, the invention relates to a measurement device for measuring the exhale capacity of a patient, whereby the measurement device comprises a main body with a through hole, which hole is arranged to receive and removably accommodate a blowing device, which blowing device is arranged so that the patient can blow through the blowing device and thereby set a rotation device, which is arranged in the blowing device, in rotary motion with a rotary speed which depends on the intensity of the blowing, wherein the measurement device further comprises an optical sensor arranged to optically sense the passage of one or several parts of the rotation device past the optical sensor as the blowing device is mounted in the hole and the rotation device rotates, and is characterised in that the measurement device comprises a cable which at a first end is connected to the main body of the measurement device and at an opposite, second, free end comprises a terminal which is arranged to be connected to a communication connection of standard type of a handheld computer unit, which handheld computer unit as such is of general type and intended for general purposes, and in that the measurement device is arranged to, when the blowing device is mounted in the hole and the rotation device rotates, from the terminal to the handheld computer unit continuously deliver a signal carrying information which directly or indirectly informs about either the instantaneous rotary speed of the rotary motion or the instantaneous air flow through the blowing device.

Furthermore, the invention relates to a software product of the type and with the characterising features according to claims 8 and 9, as well as to a method of the type and with the characterising features according to claim 12.

The invention will now be described in detail, with reference to exemplifying embodiments of the invention and to the appended drawings, wherein:

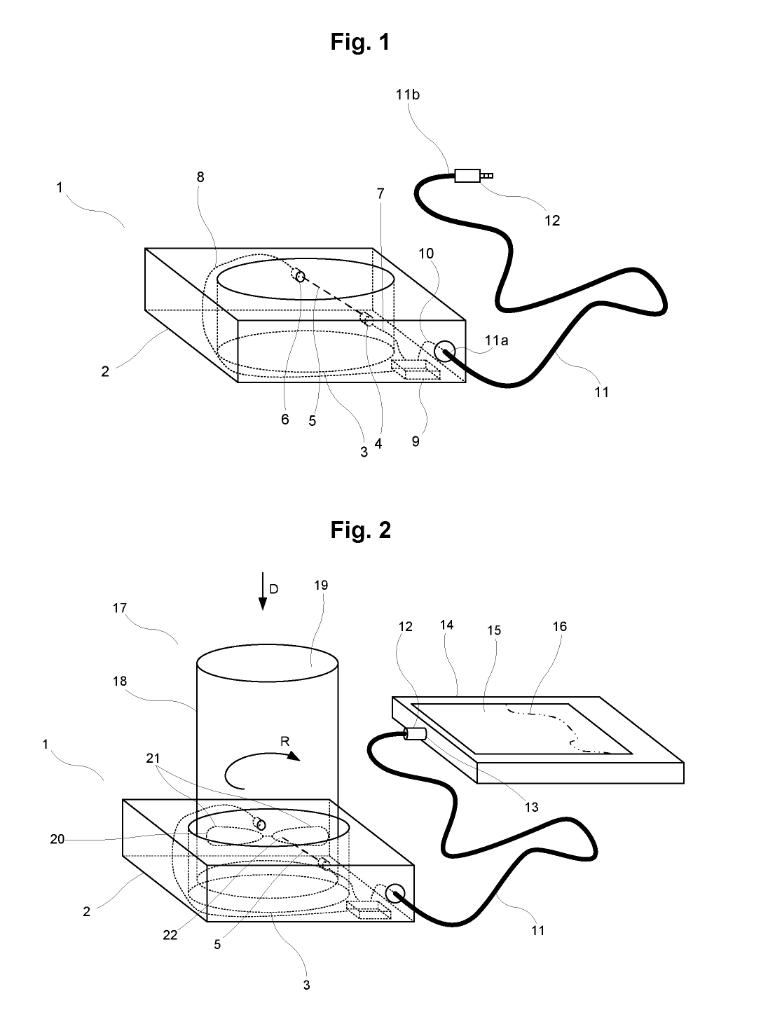

FIG. 1 schematically shows a measurement device according to the present invention;

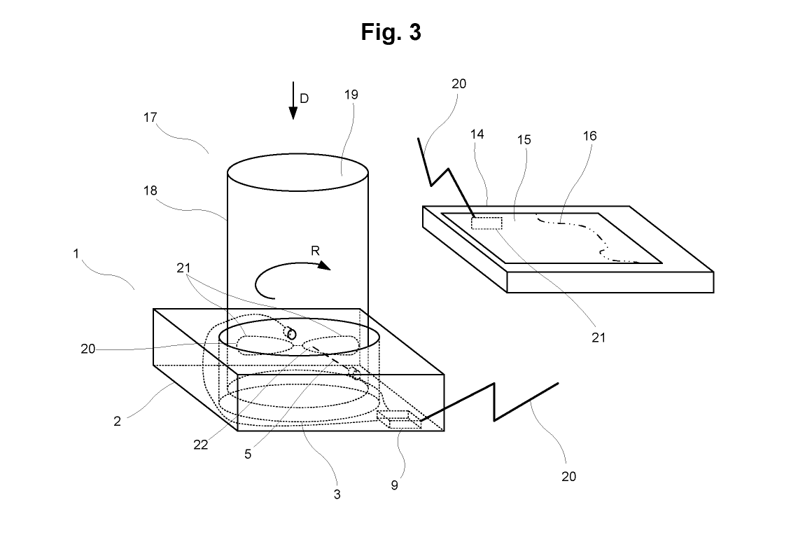

FIG. 2 schematically shows a measurement device according to the present invention together with a blowing device and a handheld computer unit, and

FIG. 3 schematically shows an alternative measurement device according to the present invention together with a blowing device and a handheld computer unit.

FIGS. 1 and 2 show a measurement device 1 according to the present invention for measuring exhale capacity of a patient. The measurement device 1 is shown, in FIGS. 1 and 2, schematically and simplified in order to increase clarity. Hidden details are shown with broken lines with short dashes.

The measurement device 1 comprises a main body 2 with a through hole 3, which hole 3 is arranged to receive and removably accommodate a blowing device 17. The blowing device 17, which is shown in FIG. 2, comprises a tube 18 which is open in both ends. A patient can blow through an opening 19 in the upper open end, and thereby set the air inside the tube 18 in motion along the longitudinal direction D of the tube 18. The blowing device 17 further comprises a rotation device 20 in the form of a small turbine or a propeller, having propeller blades 21. When the air in the tube 18 flows past the rotation device 20, it is set in rotary motion in a direction R, with a rotary speed which depends on the intensity of the patient's blowing through the opening 19.

Such blowing devices 17 are marketed by the company MIR in the USA. The tube 18 can be made of cardboard, and the lower part, comprising the rotation device 20, can be of rigid plastic. The cylinder shaped envelope surface of the tube 18 in level with the rotation device 20 is transparent, and for example made from transparent plastic.

The measurement device 1 further comprises an optical sensor 6, arranged to optically sense the passage of one or several parts of the rotation device 20, such as its propeller blades 21, past the optical sensor 6 when the blowing device 17 is mounted in the hole 3 and the rotation device 20 rotates as a result of the patient blowing through the opening 19.

Moreover, the measurement device 1 comprises an electrical cable 11, which at one end 11a is connected to the main body 2, and at its other, opposite end 11b, which is a free end, comprises a terminal 12 which is arranged to be connected to a standard communication connection 13 of a handheld computer unit 14. The handheld computer unit 14 is as such a general purpose device, intended for general purposes. In other words, the handheld unit 14 is a conventional mobile telephone, a portable computer, a tablet or any other type of unit which as such does not have as a main purpose to specifically be used together with the measurement device 1.

Neither the blowing device 17 nor the handheld computer unit 14 constitutes a part of the measurement device 1.

The optical sensor 6 is connected, via a conduit 8, to a central unit 9 in the measurement device 1, which is arranged to receive a signal from the sensor 6 and to emit, via a conduit 10 and further through the cable 11, a signal to the terminal 12.

When the blowing device 17 is mounted in the hole 3, and the patient blows so that the rotation device 20 rotates, the measurement device 1 is, according to the invention, arranged to continuously deliver, from the terminal 12 to the handheld computer unit 14, a signal carrying information which directly or indirectly informs about either the instantaneous rotary speed of the rotary motion or the instantaneous air flow through the blowing device 17.

According to a preferred embodiment, the optical sensor 6 is arranged at a first location along the periphery of the hole 3 and further arranged to sense a light signal which has been emitted towards the optical sensor 6 from a light source 4 arranged at another location, arranged at a distance from the first location, along the periphery of the hole 3, so that a light beam 5 incident towards the optical sensor 3 from the light source 4 is broken during passage of one or several parts 21 of the rotation device 17 past the optical sensor 6 as the rotation device 17 rotates. In FIG. 1, the case with an unbroken beam 5 is illustrated, in FIG. 2 the case is illustrated where the beam 5 is broken by a propeller blade 21 of the rotation device 20, in a point 22, so that the beam 5 does not arrive at the sensor 6.

It is preferred that the light source 4 emits infrared light, and that the optical sensor 3 is arranged to detect such infrared light.

The light source 4 and the optical sensor 3 are positioned in such a way so that when the rotation device 20 rotates, the light beam 5 is broken by the blades 21 one or several times for each revolution of the rotation device 20. In other words, the positioning of the source 4 and the sensor 3 is adapted to the or those types of blowing devices 17 with which the measurement device 1 is intended to be used.

The light source 4 is controlled and powered by the central unit 9, via a conduit 7.

As mentioned, the above said signal may carry information which directly or indirectly informs about either the instantaneous rotary speed of the rotary motion or the instantaneous air flow through the blowing device 17. One example of indirect information regarding instantaneous rotary speed is the case in which the signal comprises a marker for each disruption of the light beam 5. One example of a direct information regarding instantaneous rotary speed is if the signal instead carries a value of the rotary speed of the rotation device 20 as such, for instance in number of revolutions per minute. Both these examples also constitute examples of indirect information regarding instantaneous air flow, which signal in these cases then must be supplemented with a priori knowledge about the characteristics of the blowing device 17, for instance in the form of tabulated values mapping rotary speed values for the rotation device 20 against corresponding values for the air flow through the blowing device 17, in order to be able to calculate the instantaneous air flow.

According to an especially preferred embodiment, the signal which is transferred via the terminal 12 to the handheld computer unit 14 is an analogue electrical signal, and the terminal 12 is arranged to be connected, and deliver the signal, to a communication port 13 of the handheld computer unit in the form of an input arranged to receive an analog sound signal. One preferred example of such an input of the handheld computer unit 14 is a connection of audio plug or phone connector type. Suitably, the input 13 is a female connector and the terminal 12 is a male connector.

Such an audio plug connection 12, 13 is preferably of the type which is conventional as such, in which a pin, with a diameter of 2.5 mm or 3.5 mm and comprising various mutually isolated contact surfaces for different analog electrical signals along the longitudinal direction of the pin, is inserted into a corresponding sleeve for electric contacting. Such audio plugs are today normally used in handheld computer units such as mobile phones and tablets for transferring analog sound signals to and from such computer units.

Furthermore, it is preferred that the output signal from the optical sensor 6 is delivered, from the central unit 9 to the terminal 12, without intermediate digital processing, possibly also without any type of active signal processing, of the output signal. Hence, it is preferred that a raw electric voltage achieved by the sensor 6 as a response to variations in incident and detected light 5, is forwarded directly to the central unit 9, and after that directly to the cable 11 and the terminal 12 without intermediate processing. According to another exemplary embodiment, the signal may for instance be analog amplified, or be subjected to other analog signal processing such as noise reduction, before delivered to the terminal 12.

Since a measurement device 1 according to the present invention only needs to achieve a signal on the terminal 12 which is readable for the handheld computer unit 14, which in turn is intended for general purposes and therefore has a certain calculation capacity which can be used for signal processing and interpretation of the signal delivered from the measurement device 1, the measurement device 1 as such can be made very simple, and therefore cheap to manufacture. A conventional spirometer of the type described above, on the other hand, is comparatively expensive to manufacture and therefore to obtain for the individual patient.

Since many patients already have access to a handheld computer unit for general purposes, such as a mobile phone, a tablet or a portable computer, it is therefore sufficient to distribute measurement devices according to the present invention to such patients in order to achieve a complete measurement equipment for use together with blowing devices 17 of the disposable type described above, and the need for getting a special spirometer of the above described type therefore disappears.

Hence, using a measurement device comprised by a measurement device according to the invention and the user's own handheld computer equipment, the patient can perform measurements of the exhale capacity without having to obtain an expensive piece of measurement equipment or staying at a hospital. Especially, the user can achieve such measurements at home, when travelling and so forth, which caters for more frequent measurements and therefore an improved information basis for treatment strategies.

By using a terminal 12 in the form of a connection or contact of standard type, the measurement device 1 can be used together with a broad selection of different handheld computer units 14, resulting in that the measurement device 1 only has to be provided in a few, or only one single, variant(s), and still be able to achieve compatibility with a large number of different computer units 14. In the preferred case where the terminal 12 is compatible with a standard analog connection, intended to feed an audio signal to a handheld computer device, a particularly broad compatibility can be achieved, since many handheld computer units 14 comprise such a connection. In particular a connection of audio plug type is today common, and is for instance used as a microphone input socket for so called handsfree connections for mobile phones, recording input sockets for audio from auxiliary microphones and so on. Other examples of standard connections comprise so called micro-USB and the type of connection used by products sold under the trademark iPhone®.

A configuration wherein an analogue signal is delivered on the terminal 12, which for example is preferred if the terminal 12 is an audio input for instance of the audio plug type, and in particular if no digital signal processing is performed of the signal, as described above, results in that the measurement device 1 can be made very simple and therefore both cheap and reliable. For example, it is preferred that the measurement device 1 is completely free of digital electronics components.

Furthermore, it is preferred that the measurement device 1 is arranged to, via the cable 11, be connected electrically to the handheld computer unit 14 in such a way so that the measurement device 1 can be supplied with power from the connection 13 of the handheld computer unit 14. Certain digital connections can support delivery of an electric power from the connection. Even in analogue connections, such as an audio plug connection, there may be, and often is, an available voltage which may be exploited in order to provide power to such components that do not use much electricity and are necessary in the measurement device 1 for it to function.

This way, the measurement device 1 can be designed completely without other internal or external energy sources. In particular, it is preferred that the measurement device 1 does not comprise any batteries. This results in a very simple and reliable construction.

An important aspect of the present invention is the presence of the cable 11, which provides the opportunity for the patient to view a screen 15 of the computer unit 14 at the same time as the patient blows, and therefore to monitor the variation over time of the blowing intensity. To this extent, it is preferred that the cable 11 is at least 30 cm of length.

In the handheld computer unit 14, there is stored a piece of software, which software is arranged to, when executed by the computer unit, show information on a screen 15 on the handheld computer unit 14 and to the patient, concerning the exhale capacity of the patient, for instance in the form of a continuously updated graph 16 over exhale intensity as a function of time. It is also preferred that the software is arranged to administer the exhale test itself, by providing an interface using which the patient can initiate, carry through and finish a test of the exhale capacity.

The software is arranged to, when executed on the handheld computer unit 14, read and interpret the signal which arrives to a communication port 13 of the handheld computer unit 14 from the terminal 12 of the measurement device 1. Depending on the structure of the signal, it may be sufficient that the signal is interpreted information-wise by the software simply translating the signal value to instantaneous air flow. In case only indirect information regarding the air flow is available in the signal, the software is arranged to, based upon the signal value in combination with required additional data regarding the blow device 17 or the like, calculate the instantaneous air flow. For instance, this may entail that the instantaneous air flow is calculated based upon a value for the instantaneous rotary speed obtained from the signal and tabulated values, as described above. Finally, the software is arranged to show to the user the instantaneous value for a parameter which is related to the instantaneous air flow as a function of time, preferably the instantaneous or aggregated air flow itself.

In the preferred case in which the signal is an analogue signal, especially a signal which has not undergone digital signal processing, it is preferred that the software is arranged to, within the scope of these calculations, identify an instantaneous typical frequency for the signal, and to interpret this typical frequency as proportional to a rotary frequency for the rotation device 20. For example, the signal may comprise a change of a transferred voltage each time the light beam 5 is broken and/or is reconnected to the sensor 6, whereby a periodically repeated pattern accrues in the signal with a certain frequency, which frequency in that case is proportional to the rotary frequency of the rotation device 20, depending among other things upon the number of propeller blades 21. Thereafter, the software is arranged to, based upon predetermined data about the characteristics of the rotation device 20, in a way corresponding to that described above, calculate the air flow through the blowing device 17 which corresponds to the rotary frequency for the rotation device 20, in order to finally, on the screen 15, show the value for a parameter which is related to the hence calculated air flow as a function of time according to the above.

As described above, the signal can be delivered to a standard communication port of the handheld computer device 14. Depending on if the cable 11 is arranged to be connected to a digital or an analogue input of the computer unit 14, the software is arranged to, when executed, read the signal from the corresponding input. It is particularly preferred that the software is arranged to read an input of the computer unit 14 which is intended for reception and accommodation of a contact, for instance an audio plug type contact, intended for the transfer of an analogue audio signal, such as described above. This means that the software is arranged to, when executed, interpret an audio signal received by the computer unit 14 not as an audio signal but as a signal which carries information concerning the rotation of the rotation device 20, the air flow through the blowing device 17 or the like, depending on what type of signal processing is performed in the measurement device 1 before the signal is delivered to the terminal 12.

Thus, during use of the measurement device 1, firstly the blowing device 17 is inserted into the through hole 3, the cable 11 is connected to the handheld computer unit 14 and the software is initiated on the handheld computer unit 14. Thereafter, the patient blows with maximum power through the hole 19 in the blowing device 17, and thereby sets the rotation device 20 in rotary motion with a velocity that depends on the blowing intensity. Using the optical sensor 6, the measurement device 1 measures the rotary speed and delivers a signal carrying information which is sufficient for the software, with necessary background information, to be able to calculate the instantaneous air flow through the blowing device 17, to the handheld computer unit 14, via the terminal 12 which is connected to the input 13 of the computer unit 14. During the progress of the blowing, the software continuously reads the signal from the measurement device 1, and shows, after any calculations, a parameter which is related to the instantaneous air flow as a function of time, for instance in the form of a graph 16 showing instantaneous or aggregated air flow as a function of time since the beginning of blowing.

Above, a number of exemplary embodiments have been described. However, it is apparent that the skilled person can modify the described examples without departing from the basic idea of the invention.

For instance, other types of standard connections of handheld computer units may be used, such as a conventional USB-type connection.

Moreover, the information shown to the user on the screen of the computer unit may be varied, for instance by showing numbers rather than a graph, or that the display of exhale capacity data as a function of time constitutes a part of a more complex interface which also comprises another type of information.

Hence, the invention is not limited to the above described exemplary embodiments, but may be varied within the scope of the enclosed claims.

Further, as an alternative to using a cable between the measurement device and the handheld computer unit, a wireless transfer technology may be used. This is illustrated in figure 3, which is the same figure as FIG. 2 but in which the cable 11 is replaced for a wireless connection between the measurement device 1 and the handheld computer unit 14.

Hence, in this case the measurement device 1 comprises a sending device, which may constitute a part of the central unit 9 or be a separate component in the measurement device 1, which is arranged to emit a signal 20 which can be read using a standard communication connection 21 of the handheld computer unit 14.

According to a preferred embodiment, the signal is in this case sent over a standard protocol for near field communication, such as Bluetooth, Near Field Communication (NFC) and so on. According to another preferred embodiment, the measurement device 1 is arranged to wirelessly be able to communicate over the Internet or a corresponding computer network, and the computer unit 14 is then arranged to receive a signal 20 which is sent wirelessly from the measurement device 1 across such a computer network. In both these cases, the type of information regarding instantaneous rotary speed, instantaneous air flow or the like as described above is sent wirelessly via the signal 20.

1-14. (canceled)

15. Measurement device (1) for measuring the exhale capacity of a patient, whereby the measurement device comprises a main body (2) with a through hole (3), which hole is arranged to receive and removably accommodate a blowing device (17), which blowing device is arranged so that the patient can blow through the blowing device and thereby set a rotation device (20), which is arranged in the blowing device, in rotary motion with a rotary speed which depends on the intensity of the blowing, wherein the measurement device further comprises an optical sensor (6) arranged to optically sense the passage of one or several parts (21) of the rotation device past the optical sensor as the blowing device is mounted in the hole and the rotation device rotates, wherein the measurement device comprises a cable (11) which at a first end (11a) is connected to the main body of the measurement device, wherein the measurement device is arranged to, when the blowing device is mounted in the hole and the rotation device rotates, via the cable continuously deliver an analogue electric signal carrying information which directly or indirectly provides information either of the instantaneous rotary speed of the rotary motion or the instantaneous air flow through the blowing device, characterised in that the cable at a second, free end (11b), which is opposite to the first end, comprises a terminal (12) which is arranged to be connected to a communication connection (13) of standard type, in the form of an input arranged to receive an analogue audio signal, of a handheld computer unit (14), which handheld computer unit as such is a general purpose type computer unit, intended for general purposes.

16. Measurement device (1) according to claim 15, characterised in that the optical sensor (6) is arranged at a first location along the periphery of the hole (3) and further arranged to sense a light signal which is emitted towards the optical sensor from a light source (4) arranged at another, second location, arranged at a distance from the first location along the periphery of the hole, so that a light beam (5) incident towards the optical sensor from the light source is broken during passage of one or several parts (21) of the rotation device (20) past the optical sensor as the rotation device rotates.

17. Measurement device (1) according to claim 15, characterised in that the terminal (12) is in the form of an audio plug.

18. Measurement device (1) according to claim 15, characterised in that the output signal from the optical sensor (6) is delivered to the terminal (12) without intermediate digital processing of the output signal.

19. Measurement device (1) according to claim 15, characterised in that the cable (11) is at least 30 cm of length.

20. Measurement device (1) according to claim 15, characterised in that the measurement device is arranged to be connected electrically, via the cable (11), to the handheld computer unit (14) in such a way so that the measurement device can be powered only by the use of electrical energy from the handheld computer unit.

21. Software for execution on a handheld general purpose computer unit (14), and arranged to, on a display (15) of the handheld computer unit and to a patient, display information regarding the exhale capacity of the patient, characterised in that the software is arranged to
read an analogue electrical signal arriving to a communication port (13) of the handheld computer unit from a measurement device (1) according to claim 15, which communication port is in the form of an input arranged to receive an analogue audio signal and which signal carries information which directly or indirectly gives information either about the instantaneous rotary speed of a rotation device (20) in a blowing device (17) which is mounted in the measurement device or the instantaneous air flow through such a blowing device;
if needed, calculate the instantaneous air flow based upon the instantaneous rotary speed of the said rotation device; and
on a display (15) of the handheld computer unit, show the value of a parameter which is related to the instantaneous air flow as a function of time.

22. Software for execution on a handheld general purpose computer unit (14), and arranged to, on a display (15) of the handheld computer unit and to a patient, display information regarding the exhale capacity of the patient, which software is arranged to read a signal, to identify an instantaneous typical frequency of the said signal, to interpret the typical frequency as being proportional to a rotary frequency for a rotation device (20) being subjected to an air flow setting the rotating device in rotary motion, to calculate the air flow based upon predetermined data about the properties of the rotation device, and to on a display (15) of the handheld computer unit show the value of a parameter which is related to the thus calculated air flow as a function of time, characterised in that the signal is an analogue electric signal which arrives to a communication port (13) of the handheld computer unit, which communication port is in the form of an input arranged to receive an analogue audio signal.

23. Software according to claim 22, characterised in that the communication port (13) is arranged to receive and accommodate an audio plug.

24. Method for measuring the exhale capacity of a patient, whereby a blowing device (17), comprising a rotation device (20) arranged to be put in rotary motion by an air flow which is achieved through the blowing device, is inserted into a through hole (3) of a measurement device (1), after which the patient blows through the blowing device and thereby sets the rotation device in rotary motion with a speed which depends on the intensity of the blowing, whereby an optical sensor (6) in the measurement device is caused to sense the passage of one or several parts (21) of the rotation device past the optical sensor, whereby a software in the handheld computer unit is caused to read a signal, if so is needed to calculate the instantaneous air flow based upon the instantaneous rotary speed, and on a display (15) of a handheld general purpose computer unit (14) show the value for a parameter which is related to the instantaneous air flow as a function of time, characterised in that, while the blowing is ongoing, the measurement device is caused to, via a cable (11) which at a first end (11a) is connected to the measurement device and at a second, free end (11b) comprises a terminal which is arranged to be connected to a communication connection (13) of standard type, in the form of an input arranged to receive an analogue audio signal, of the handheld computer unit, to the communication connection of the handheld computer unit continuously deliver an analogue electric signal carrying information which directly or indirectly gives information either about the instantaneous rotary speed of the rotary motion or the instantaneous air flow through the blowing device.

25. Method according to claim 24, characterised in that the terminal (12) is in the form of an audio plug.

26. Method according to claim 24, characterised in that the output signal from the optical sensor (6) is delivered to the terminal (12) without intermediary digital processing of the output signal.

27. Method according to claim 24, characterised in that the measurement device (1) is powered completely via the cable (11), which is connected electrically to the handheld computer unit (14) and thereby receives electric energy from the handheld computer unit.

28. Method according to claim 24, characterised in that the software is arranged to identify an instantaneous typical frequency of the signal, to interpret the typical frequency as being proportional to a rotary frequency for the rotation device (20) and to calculate the air flow based upon predetermined data regarding the properties of the rotation device.
